(19) United States
(12) Patent Application Publication
(10) Pub. No.: US 2022/0403588 A1
(43) Pub. Date: Dec. 22, 2022

(54) THE CHEMICAL COMPOSITION FOR IMPREGNATION VIA SPRAYING ON DISPOSABLE SUGICAL MASKS, SLEEVES GOWNS MADE FROM HYDROPHILIC AND HYDROPHOBIC MATERIALS FOR THE INACTIVATION OF BACTERIA AND VIRUSES SUCH AS CORONAVIRUS, SARS, MERS, INFLUENZA A AND INFLUENZA B VIRUSES

(71) Applicant: Cheng KIT YEW, Singapore (SG)

(72) Inventors: Cheng KIT YEW, Singapore (SG); Benjamin VALDEZ SALAS, Mexicali (MX)

(21) Appl. No.: 17/614,079

(22) PCT Filed: Mar. 12, 2020
(86) PCT No.: PCT/SG2020/050131

§ 371 Date: Nov. 24, 2021

(30) Foreign Application Priority Data
Mar. 9, 2020 (SG) 10202002135U

(51) Int. Cl. D06M 16/00; D06M 11/83; D06M 13/00

(52) U.S. Cl. CPC D06M 16/003; D06M 11/83; D06M 13/005

(57) ABSTRACT

This invention relates to the Chemical Composition for impregnation via spraying on disposal surgical masks and sleeved gowns made from hydrophilic and hydrophobic materials to inactivate bacteria and viruses, comprising of colloidal silver nanoparticles, Isopropanol Alcohol/Ethanol Alcohol Chlorhexidine/Quaternary Ammonium Compound, Peppermint/Spearmint Essential oils and water.

This invention relates to the Chemical Composition for impregnation via spraying on disposal surgical masks and sleeved gowns made from hydrophilic and hydrophobic materials to inactivate bacteria and enveloped viruses such as Coronavirus, SARS, MERS, Influenza A and Influenza B Viruses comprising of aqueous colloidal silver nanoparticles, Isopropanol Alcohol/Ethanol Alcohol Chlorhexidine gluconate/Quaternary Ammonium Compound (alkyl dimethyl benzyl ammonium chloride, alkyl didecyl dimethyl ammonium chloride, and dialkyl dimethyl ammonium chloride), Peppermint/Spearmint Essential oils and water.

### DESCRIPTION OF PRIOR ART

Disposable surgical face masks and sleeved gowns are the most widely used by healthcare professional and the public during an epidemic or pandemic. Disposable surgical masks and sleeved gowns made from hydrophilic and hydrophobic materials prevent the bacteria and viruses from infecting the healthcare professionals and the spread of diseases as it helps catch the bacteria shed in liquid droplets during sneezing into the wearer's mouth and nose.

It has long been the object of the applicants' research to provide the chemical composition for impregnation via spraying of surgical masks and sleeved gowns to protect healthcare professionals against bacteria and enveloped virus such as Coronavirus, SARS, MERS, Influenza A and Influenza B Viruses infection while using said gears.

Primarily disposable surgical masks and sleeved gowns have traditionally been made from nonwoven fabrics and polypropylene with hydrophobic properties or a water chemical-repellent chemical finish or other hydrophilic materials.

US Patent No. 20140377467 A1 discloses A method for inhibiting the spread of nosocomial infections in institutional health care settings comprises treating outer garments, worn indoors by employed staff of the institution, to impart antimicrobial properties to those garments by immersing the garments in a solution of glyxol, eugenol and water, squeezing the solution out of the garments, curing the wetted garments under heat, and drying the cured garments; and thereafter requiring employed staff to wear the treated garments while working at the institution; laundering the garments after being worn by the staff, for further wear by the staff, and requiring employed staff to wear the treated garments after the garments have been laundered for so long as the garments retain their antimicrobial properties.

It is different from applicants' invention of chemical composition for impregnation via spraying on disposal surgical masks and sleeved gowns made from hydrophilic and hydrophobic materials.

No disclosure is included for aqueous colloidal silver nanoparticles, isopropanol/ethanol alcohol, peppermint/spearmint essential oils and chlorhexidine gluconate/quaternary ammonium compound (alkyl dimethyl benzyl ammonium chloride, alkyl didecyl dimethyl ammonium chloride, and dialkyl dimethyl ammonium chloride) in US Patent No. 20140377467 A1 with respect of using it for impregnation of disposable surgical masks and sleeved gowns made from hydrophilic and hydrophobic materials.

The said patent fails to recognize the need of using aqueous colloidal silver nanoparticles for good bactericidal residual effect and the synergistic use of chlorhexidine gluconate/quaternary ammonium compound (alkyl dimethyl benzyl ammonium chloride, alkyl didecyl dimethyl ammonium chloride, and dialkyl dimethyl ammonium chloride) with Isopropanol/Ethanol Alcohol as co-disinfectants and to increase the hydrophilic properties of essential oils.

The said patent also fails to recognize the use peppermint/spearmint essential oils to enhance its antimicrobial properties of the impregnated surgical masks and sleeved gowns. The applicant's invention is nonobvious even to a person skilled in the art of US Patent No. 20140377467 A1.

WIPO Patent No. W02012001702 A1 discloses A formulation provided which comprises at least one phenolic compound in an amount of about 0.5 to about 1.2% of the mass of the formulation; at least one fragrance in an amount of about 0.3 to about 1.0% of the mass of the formulation; and at least one alcohol in an amount of about 90 to about 99% of the mass of the formulation. Said formulation is surfactant free and being capable of rendering anti-microbial finish to fabrics. And a process for preparing a fabric with anti-microbial finish is provided.

It is different from applicants' invention of chemical composition for impregnation via spraying on disposal surgical masks and sleeved gowns made from hydrophilic and hydrophobic materials, no disclosure is included for aqueous colloidal silver nanoparticles, isopropanol/ethanol alcohol, peppermint/spearmint essential oils and chlorhexidine gluconate/quaternary ammonium compound (alkyl dimethyl benzyl ammonium chloride, alkyl didecyl dimethyl ammonium chloride, and dialkyl dimethyl ammonium chloride), in WIPO Patent No. WO2012001702 A1.

The said patent fails to recognize the need of using aqueous colloidal silver nanoparticles for good bactericidal residual efficacy and the synergistic use of chlorhexidine gluconate/Quaternary Ammonium Compound with Isopropanol/Ethanol Alcohol.

The said patent also fails to recognize the use peppermint/spearmint essential oils to enhance its antimicrobial properties of the impregnation via spraying on surgical masks and sleeved gowns. The use toxic phenolic compound is not in the applicants' invention. Phenol is highly irritating to the skin, eyes, and mucous membranes in humans after acute (short-term) inhalation or dermal exposures.

Phenol is considered quite toxic to humans via oral exposure. Anorexia, progressive weight loss, diarrhea, vertigo, salivation, a dark coloration of the urine, and blood and liver effects have been reported in chronically (long-term) exposed humans. Animal studies have reported reduced foetal body weights, growth retardation, and abnormal development in the offspring of animals exposed to phenol by the oral route.

The applicants' Chemical Composition does not include the use of phenol. The applicant's invention is nonobvious even to a person skilled in the art of WIPO Patent No. WO2012001702

U.S. Pat. No. 2,791,518 discloses A method of imparting microbicidal properties to articles such as textiles by immersing the article in a first aqueous solution containing a water-soluble basic nitrogen compound (ammonia) and a monovalent silver salt soluble in said solution, followed by a second immersion in a second solution containing a second salt capable of ion exchange with the silver salt such that a monovalent silver salt precipitate is formed within the article. The formed silver precipitate is sparingly water soluble and imparts microbicidal properties to the articles so treated.

It is not related to the applicants' invention for the chemical composition for impregnation via spraying on disposal surgical masks and sleeved gowns made from hydrophilic and hydrophobic materials, no disclosure of the chemical composition for aqueous colloidal silver nanoparticles, isopropanol/ethanol alcohol, peppermint/spearmint essential oils and chlorhexidine gluconate/Quaternary Ammonium Compound (alkyl dimethyl benzyl ammonium chloride, alkyl didecyl dimethyl ammonium chloride, and dialkyl dimethyl ammonium chloride), in U.S. Pat. No. 2,791,518 with respect of using it for impregnation of disposable masks and gowns.

Ammonia is an irritant and the solution and gas can cause burns of the skin, eyes, mouth, and lungs. When mixed with household bleach (sodium hypochlorite solution) it forms toxic and explosively unstable chloramines.

The applicants' chemical composition does not include the use water-soluble nitrogen compound (ammonia).

The applicant's invention is nonobvious even to a person skilled in the art of U.S. Pat. No. 2,791,518.

U.S. Pat. No. 6,979,491 B2 discloses the invention a yarn with antimicrobial effects. The antimicrobial antifungal effect of the yarn is derived from nanosilver particles (diameter between 1 and 100 nm) which are adhered to the yarn. The yarn contains fibre which are made of cotton, linen, silk, wool, leather, blending fabric, synthetic fibre, or any combination thereof. The yarn can be used to make cloth to be used particularly for treating patients with burns or wound. The cloth made from the antimicrobial yarn can be further used to make clothes such as under wears, socks, shoe cushions, shoe linings, bed sheets, pillowcases, towels, women hygiene products, laboratory coats, and medical robes. The present invention also provides a method for making the antimicrobial yarn.

The said patent totally differs from the applicants' invention of chemical composition for impregnation via spraying on disposal surgical masks and sleeved gowns made from hydrophilic and hydrophobic materials, no disclosure of the chemical composition of aqueous colloidal silver nanoparticles, isopropanol/ethanol alcohol, peppermint/spearmint essential oils, chlorhexidine gluconate/Quaternary (alkyl dimethyl benzyl ammonium chloride, alkyl didecyl dimethyl ammonium chloride, and dialkyl dimethyl ammonium chloride), in U.S. Pat. No. 6,979,491 B2.

The applicants' invention used said chemical composition for impregnation via spraying on disposal surgical masks and gowns made from hydrophilic and hydrophobic materials to inactivate bacteria and enveloped viruses such as Coronavirus, SARS, MERS, Influenza A and Influenza B viruses is not claimed under the U.S. Pat. No. 6,979,491 B2.

The applicant's invention is nonobvious even to a person skilled in the art of U.S. Pat. No. 6,979,491 B2.

U.S. Pat. No. 5,271,952 discloses A method of treating fibres to render them electrically conductive as well as anti-bacterial comprising immersing the fibres in a bath comprising an aqueous solution of a source of divalent copper ions, a reducing agent, sodium thiosulfate and a source of iodide ions, whereby copper iodide is adsorbed into the fibres.

It totally differs from applicants' invention applicants' invention of chemical composition for impregnation via spraying on disposal surgical masks and sleeved gowns made from hydrophilic and hydrophobic materials, no disclosure of the applicants' chemical composition of aqueous colloidal silver nanoparticle, isopropanol/ethanol alcohol, peppermint/spearmint essential oils and chlorhexidine gluconate/Quaternary Ammonium Compound Quaternary (alkyl dimethyl benzyl ammonium chloride, alkyl didecyl dimethyl ammonium chloride, and dialkyl dimethyl ammonium chloride) in U.S. Pat. No. 5,271,952 with respect of using it for impregnation via spraying on disposable masks and gowns to inactivate bacteria and enveloped viruses such as Coronavirus, SARS, MERS, Influenza A and Influenza B.

The applicant's invention is nonobvious even to a person skilled in the art of U.S. Pat. No. 5,271,952.

U.S. Patent No. 200601448 A1 discloses Antimicrobial fibres useful for the manufacture of gas permeable fabrics are manufactured by co-extrusion of polymerics and antimicrobial materials. The fibres may be ed in various combinations to provide antimicrobial fabric materials.

It differs from applicants' invention of the chemical composition for impregnation via spraying on disposal surgical masks and sleeved gowns made from hydrophilic and hydrophobic materials, no disclosure of chemical composition comprising of aqueous colloidal silver nanoparticles, isopropanol/ethanol alcohol, peppermint/spearmint essential oils and chlorhexidine gluconate/Quaternary Ammonium Compound (alkyl dimethyl benzyl ammonium chloride, alkyl didecyl dimethyl ammonium chloride, and dialkyl dimethyl ammonium chloride) and water in Patent No. 200601448 A 1 with respect of using it for impregnation via spraying on disposable masks and gowns made from hydrophilic and hydrophobic materials for inactivation of bacteria and enveloped viruses such as Coronavirus, SARS, MERS, Influenza A and Influenza B.

The applicant's invention is nonobvious even to a person skilled in the art of U.S. Patent No. 200601448 A1.

US Patent No. 20080092909 A1 discloses the invention a face mask that is adapted to fit over the nose, mouth and chin of the wearer and to conform at its edges reasonably closely to the face and neck of the wearer. The mask may also include an eye shield. It is not related to the applicants' invention of the Chemical Composition for impregnation via spraying on disposal surgical masks and sleeved gowns made from hydrophilic and hydrophobic materials to inactivate bacteria and enveloped viruses such as Coronavirus, SARS, MERS, Influenza A and Influenza B, no disclosure of chemical composition comprising of aqueous colloidal silver nanoparticles, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride) and peppermint/spearmint essential oils and water in U.S. Patent no. 20080092909 A1.

The applicants' invention is non-obvious even to a person skilled in the art of US Patent No. 20080092909 A1.

U.S. Pat. No. 7,462,753 (Ma) discloses A nano-silver wound dressing consisting of a skin contact layer made from hydrophilic cloth and directly contacting a wound on the surface of the skin, a disinfecting (or bactericidal) antitoxic layer made from activated charcoal cloth impregnated with nanocrystalline silver, a blood absorbing and styptic layer made from a superabsorbent polymer non-woven cloth, an isolation layer made from a composite fabric with a pore size of less than 5 micrometers and an elastic bandage for fixing a main body on the site of wound.

It is not related to applicants' invention of the Chemical Composition for impregnation via spraying on surgical masks and sleeved gowns made from both hydrophilic and hydrophobic materials to inactivate bacteria and enveloped viruses such as Coronavirus, SARS, MERS, Influenza A and Influenza B Viruses, comprising of aqueous colloidal silver nanoparticles, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), and peppermint/spearmint essential oils and water in U.S. Pat. No. 7,462,753 (Ma).

The applicants' invention is non-obvious even to a person skilled in the art of U.S. Pat. No. 7,462,753 (Ma).

U.S. Pat. No. 6,967,261 (Soereus) discloses A bandage or wrap with antibiotics therein. It is further described that an included nonwoven material may be treated to be hydrophilic or may include superabsorbent materials. It is not related to applicants' invention of the Chemical Composition for impregnation via spraying on surgical masks and sleeved gowns made from both hydrophilic and hydrophobic materials to inactivate bacteria and enveloped viruses such as Coronavirus, SARS, MERS, Influenza A and Influenza B. No disclosure of chemical composition comprising of aqueous colloidal silver nanoparticles, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), peppermint/spearmint essential oils and water in U.S. Pat. No. 6,967,261.

The applicants' invention is non-obvious even to a person skilled in the art of U.S. Pat. No. 6,967,261

U.S. Patent Application No. 2007/0044801A discloses Face mask for reducing the number of microbes to which a wearer is exposed is provided. The face mask includes a body portion that has an outer layer that has been treated with a germicidal agent in an effective amount. The layer may be a nonwoven fabric like a spurn-bond, melt-blown or conform layer and may be a laminate of such layers. The face mask having such a germicidal treatment can result in a reduction in microbial activity as compared to another face mask, identical but for the germicidal agent.

It is not related to applicants' invention of the Chemical Composition for impregnation via spraying on surgical masks and sleeved gowns made from both hydrophilic and hydrophobic materials to inactivate bacteria and enveloped viruses such as Coronavirus, SARS, MERS, Influenza A and Influenza B, no disclosure is included for aqueous colloidal silver nanoparticles, isopropanol/ethanol alcohol, peppermint/spearmint essential oils, chlorhexidine gluconate/Quaternary Ammonium Compound (alkyl dimethyl benzyl ammonium chloride, alkyl didecyl dimethyl ammonium chloride, and dialkyl dimethyl ammonium chloride), and water in US Patent Application No. 2007/0044801 A with respect of using it for impregnation of disposable masks and gowns made from both hydrophilic and hydrophobic materials.

The said patent fails to recognize the need of using aqueous colloidal silver nanoparticles for good bactericidal residual efficacy and the synergistic use of chlorhexidine gluconate/Quaternary Ammonium Compound (alkyl dimethyl benzyl ammonium chloride, alkyl didecyl dimethyl ammonium chloride, and dialkyl dimethyl ammonium chloride), with Isopropanol Alcohol.

The said patent also fails to recognize the use peppermint/spearmint essential oils to enhance its antimicrobial properties of the impregnation via spraying on disposal surgical masks and sleeved gowns.

The applicant's invention is nonobvious even to a person skilled in the art of US Patent No. 2007/0044801A.

WIPO Patent No. W02005012630 A2 discloses the methods for producing antimicrobial ballistic fabrics and protective apparel comprising aramid, polybenzazole or high-performance polyethylene, which articles are rendered antimicrobial by a chitosan agent, thereby preventing the development of odour and fungal and bacterial growth.

The said patent totally differs from applicant's invention of the Chemical Composition for impregnation via spraying on surgical masks and sleeved gowns made from both hydrophilic and hydrophobic materials to inactivate bacteria and enveloped viruses such as Coronavirus, SARS, MERS, Influenza A and Influenza B, no disclosure is included for colloidal silver nanoparticle, isopropanol/ethanol alcohol, essential oils, chlorhexidine gluconate and water in WIPO Patent No. W02005012630 A 2 with respect of using it for impregnation of disposable masks and gown made from hydrophilic and hydrophobic materials.

The said patent fails to recognize the need of using aqueous colloidal silver nanoparticles for good bactericidal residual efficacy and the synergistic use of chlorhexidine gluconate/Quaternary Ammonium Compound (alkyl dimethyl benzyl ammonium chloride, alkyl didecyl dimethyl ammonium chloride, and dialkyl dimethyl ammonium chloride) with Isopropanol/Ethanol Alcohol.

The said patent also fails to recognize the use peppermint/spearmint essential oils to enhance its antimicrobial properties of the impregnation via spraying on disposal surgical masks and sleeved gowns made from hydrophilic and hydrophobic materials.

The applicant's invention is nonobvious even to a person skilled in the art of WIPO Patent No. W02005012630 A2.

China Patent No. CN 102144817 A discloses A protective facial mask containing nano silver and chitosan and a production method of the protective facial mask. The protective facial mask provided by the invention is composed of five gauze layers, wherein two of the five gauze layers are functional absorbent cotton gauzes containing the nano silver and the chitosan. The protective facial mask has the functions of antibiosis, eliminating unusual smell and absorbing dust, and can effectively prevent pathogenic bacteria or virus from invading into a human body through the oral cavity and the nasal cavity so as to prevent communicable diseases; and the protective facial mask is excellent in air permeability. The production method of the protective facial mask is simple, wide in material source and suitable for industrialized production.

It is not related to applicants' invention of the Chemical Composition for impregnation via spraying on disposal surgical masks and sleeved gowns made from both hydrophilic and hydrophobic materials to inactivate bacteria and enveloped viruses such as Coronavirus, SARS, MERS, Influenza A and Influenza B no disclosure of chemical composition comprising of aqueous colloidal silver nanoparticles, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), peppermint/spearmint essential oils and water in China Patent No. CN 102144817.

The applicant's invention does not include chitosan which is a linear polysaccharide composed of randomly distributed β-linked D-glucosamine and N-acetyl-D-glucosamine as claimed by China Patent No. CN 102144817 A.

The applicants' invention is non-obvious even to a person skilled in the art of China Patent No. 102144817 A.

China Patent CN 104366754 A discloses the invention of an antibacterial dustproof health mask with water lily fibre wadding. The antibacterial dustproof health mask is characterized in that water lily whole-plant fibre, honeysuckle whole-plant fibre and mint leaf fibre are provided, the water lily fibre is carded into uniform fibre wadding by processes, such as cleaning and rolling, carding and laying, and needling, the uniform fibre wadding is repeatedly folded into a wadding layer 0.5-1 cm thick, the wadding is finished in an interlayer of mask nonwoven filter material fabric, the middle of the fabric is in a five-fold laminated structure which can be extended in the vertical direction to fit the face, and a soft embedded bridge rib made of water lily leaf is embedded in the wing parts of the mask, allowing the wing parts to be conveniently pressed to fit the bridge of the face.

It is not related to applicants' invention of the Chemical Composition for impregnation via spraying on disposal surgical masks and sleeved gowns made from both hydrophilic and hydrophobic materials to inactivate bacteria and enveloped viruses such as Coronavirus, SARS, MERS, Influenza A and Influenza B viruses and no disclosure is included for aqueous colloidal silver nanoparticles, isopropanol/ethanol alcohol, peppermint/spearmint essential oils and chlorhexidine gluconate/Quaternary Ammonium Compound (alkyl dimethyl benzyl ammonium chloride, alkyl didecyl dimethyl ammonium chloride, and dialkyl dimethyl ammonium chloride) in China Patent No. 104366754 A.

The applicants' invention is non-obvious even to a person skilled in the art of China Patent No. 10366754 A.

China Patent No. CN 101700040 A discloses A nano-silver anti-bacterial liquid, a nano-silver anti-bacterial fabric containing the same and a preparation method thereof. The nano-silver anti-bacterial liquid comprises nano-silver particles, organic fluorins polymers and nano titanium dioxide particles. The invention also provides the nano-silver anti-bacterial fabric containing the nano-silver anti-bacterial liquid and the preparation method thereof. The nano-silver anti-bacterial liquid has the advantages of small particle diameter, good dispersity, good stability and the like. The prepared nano-silver anti-bacterial fabric and the preparation method thereof have the advantages of uniform distribution of nano-silver particles in the fabric, low cost and easy industrialization.

It is not related to applicants' invention of the Chemical Composition for impregnation via spraying on disposal surgical masks and sleeved gowns made from both hydrophilic and hydrophobic materials to inactivate bacteria and enveloped viruses such as Coronavirus, SARS, MERS, Influenza A and Influenza B viruses.

The applicant's invention does not include the use of fluorins polymers and nano titanium dioxide. Nano Titanium Dioxide is classified as “possibly carcinogenic” by International Agency for Research on Cancer (IARC) based on studies that showed increased lung tumours in rats associated with titanium dioxide inhalation particles.

The said patent consists of organic fluorins polymers and titanium dioxide particles are not claimed by the applicants. No disclosure is included for isopropanol/ethanol alcohol, peppermint/essential oils and chlorhexidine gluconate/Quaternary Ammonium Compound (alkyl dimethyl benzyl ammonium chloride, alkyl didecyl dimethyl ammonium chloride, and dialkyl dimethyl ammonium chloride) in China Patent CN 101700040A with respect of using it for impregnation of disposable masks and gowns made from both hydrophilic and hydrophobic materials.

The said patent fails to recognize the need of using chlorhexidine gluconate with isopropanol/Ethanol Alcohol as co-disinfectants.

The said patent also fails to recognize the use peppermint/spearmint essential oils to enhance its antimicrobial properties of the impregnation via spraying on disposal surgical masks and sleeved gown made from hydrophilic and hydrophobic materials. Hence, the use of chemical composition by the applicants is nonobvious even to a person skilled in the art of US Patent No. 20140377467 A1.

All prior art references cited herein differ from applicants' invention of The Chemical Composition for impregnation of surgical masks and sleeved gowns made from hydrophilic and hydrophobic materials for inactivation bacteria and viruses, comprising of aqueous silver nanoparticles, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), Peppermint/Spearmint essential oils and water.

### BACKGROUND OF INVENTION

Infectious diseases are a continuing threat, with constant emergence or re-emergence of serious diseases in various parts of the world and healthcare workers (HCWs) are particularly at-risk of exposure. Various types of personal protective equipment (PPE) are recommended and used by HCWs to protect from infections, including medical masks, respirators, gloves, gowns, goggles and face shield. In healthcare settings, medical masks are used by HCWs to protect from splashes and sprays of blood and body fluids, and by sick individuals to prevent spread of respiratory infections to others. Reuse and extended use of masks are also common in many parts of the world, particularly during outbreaks and pandemics. Respiratory pathogens may be present on used masks layers and lead to infection of the wearer

In hospital settings, these pathogens may be generated from breathing, coughing or sneezing patients or during aerosol generating medical procedures. Studies have shown that influenza virus can remain airborne for 3 hours after a patient has passed through an emergency department. While using masks, or during long periods of time of re-using them, these pathogens may cause infection through hand or skin contamination, ingestion, or mucus membrane contact.

The use of surgical facemasks and sleeved gowns are ubiquitous whenever there is an epidemic or more so in a pandemic outbreak. Facemasks have long been thought to confer protection to the users, patients from wound infection and contamination from the operating surgeon and other members of the healthcare staff. It is used as a physical barrier to protect the user from hazards, such as splashes of large droplets of body fluids. Surgical masks also protect other people against infection from the person wearing the surgical mask

Contamination with viruses and bacteria on surgical facial masks and sleeved gowns by different people in hospitals are highly probable. Using of the disposable clothing products and personal protective equipment with antimicrobial finishing can provide a good protection against transmission of diseases for both the healthcare professionals and the patients. A surgical mask, sleeved gown are designed to be worn by healthcare professionals during surgery and during nursing to catch the bacteria shed in liquid droplets and aerosols from the health professionals' mouths and noses. They are not designed to kill viruses and bacteria. They are less effective than respirators, such as N95 or FFP masks, which provide better protection due to their material, shape and tight seal. Using appropriate protective clothing is possible to create a barrier to eliminate or reduce contact and droplet exposure, and therefore prevent the transfer of microorganisms between patients and healthcare professionals.

Avoidance of Inhaling airborne bacteria or virus particles during an epidemic or pandemic situation is of paramount importance for everybody especially healthcare professionals. Contamination of surgical masks, sleeved gowns and other protective gears with viruses and bacteria will result in dire consequences among wearers of such gears. To protect healthcare professionals and users of said gears, the applicants see such a critical need to develop a disinfectant that fortifies with silver nanoparticles for impregnation of said gears with good residual effects to inactivate any enveloped viruses and bacteria landed on facial masks and gowns. In view of the above a potent chemical composition against enveloped viruses/bacteria consisting of aqueous colloidal silver nanoparticle, isopropanol/ethanol alcohol, chlorhexidine glucnate, peppermint/spearmint essential oils with colloidal silver nanoparticle was hence developed.

It has long been the object of applicants' research to provide the chemical composition to disinfect surgical masks and sleeved gowns of healthcare professionals via impregnation of said chemical composition into disposal surgical masks and sleeved gowns to inactivate bacteria, enveloped viruses such as Coronavirus, SARS, H5N1 MERS, Influenza A and Influenza B Viruses.

### SUMMARY OF THE INVENTION

The present invention has been made to solve the above-described problems of inactivating bacteria and enveloped viruses such as Coronavirus, SARS, MERS, Influenza A and Influenza B viruses on disposal surgical masks and sleeved gowns wore by healthcare professionals and the public at large during an epidemic or pandemic outbreak.

The chemical composition comprising of aqueous colloidal silver nanoparticle, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), Peppermint/Spearmint Essential Oils and water.

This invention relates to the chemical composition for impregnation via spraying on disposal surgical masks and sleeved gowns to inactivate bacteria and enveloped viruses such as Coronavirus, SARS, MERS, Influenza A and Influenza B Viruses, comprising of 5-30% by weight of aqueous colloidal silver nanoparticles with size ranges from 2-150 nm, 50-80% by weight Isopropanol Alcohol/Ethanol Alcohol, 0.1-8% by weight of Chlorhexidine gluconate/Quaternary Ammonium Compound (alkyl dimethyl benzyl ammonium chloride, alkyl didecyl dimethyl ammonium chloride, and dialkyl dimethyl ammonium chloride), 0.1-5% by weight Peppermint/Spearmint Essential oils and 2-17% by weight of water.

In an embodiment of the invention, aqueous colloidal silver nanoparticles with size ranges from 2-150 nm is used to increase the residual efficacy of the disinfectant impregnated into disposal surgical masks and gowns. The extremely small size of silver nanoparticles results in the particles having a large surface area relative to their volume. This allows them to easily interact with other particles and increases their antibacterial efficiency. This effect can be so great that one gram of silver nanoparticles is all that is required to give antibacterial properties to hundreds of square metres of substrate material. Hence, making it commercially viable for impregnation of disposal of surgical gowns and sleeved gowns. Silver's antimicrobial effect has been demonstrated in numerous applications against different types of microorganisms. The bactericidal efficacy of silver is through its binding to disulphide or sulfhydryl groups in cell wall proteins of bacteria, silver nanoparticle also binds to DNA. Through these binding events, metabolic processes are disrupted, leading to cell death.

In another embodiment Isopropanol/Ethanol Alcohol is added to the chemical composition for inactivating enveloped viruses via denaturation of the capsid protein and killing bacteria by destroying peptidoglycan/lipopolysaccharide cell wall of the bacteria. It also enhances the hydrophilic properties of peppermint/spearmint essential oils used in the chemical formulation.

In another embodiment, Chlorhexidine gluconate/Quaternary Ammonium Compound (alkyl dimethyl benzyl ammonium chloride, alkyl didecyl dimethyl ammonium chloride, and dialkyl dimethyl ammonium chloride) is used as a disinfectant that would rapidly destroy bacteria, fungi, viruses, and protozoans. Chlorhexidine is the preferred choice due to its a broad-spectrum biocide property and its effectiveness against Gram-positive bacteria, Gram-negative bacteria and fungi. It has both bacteriostatic (inhibits bacterial growth) and bactericidal (kills bacteria) making it an excellent choice for the chemical composition used for the impregnation disposal surgical masks and sleeved surgical gowns.

In a further embodiment, Peppermint/Spearmint essential oils is used to enhance the antimicrobial properties of the said chemical composition. Peppermint essential oil is the preferred essential oil because of its antimicrobial effects against both Gram-positive and Gram-negative bacteria. Peppermint essential oil is also found to possess antiviral and fungicidal activities.

In another further embodiment, water is used as a catalyst and has a major role in denaturing the capsid proteins of the enveloped viruses and cell membranes of bacteria. It also serves a carrier of all the above-mentioned ingredients of the said chemical composition.

The above said invented chemical formulation makes disposal surgical masks and sleeved gowns safer for healthcare professional and public during an epidemic or pandemic outbreak was not mentioned in US Patent No. 20140377467 A1, WIPO Patent No. W02012001702 A1, U.S. Pat. No. 2,791,518, U.S. Pat. No. 6979491 B2, U.S. Pat. No. 5,271,952, U.S. Patent No. 200601448 A1, U.S Patent No. 20080092909 A1, U.S. Pat. No. 7,462,753 (Ma), U.S. Pat. No. 6,967,261, U.S. Patent Application No. 2007/0044801A, WIPO Patent No. 102005012630 A2, China Patent No. CN 102144817 A, China Patent CN 104366754 A, and China Patent No. CN 101700040 A.

### DETAILED DESCRIPTION OF THE PREFERRED EMBODIMENTS

The following descriptions are exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

According to the aspect of the present invention, it is the object of the invention to provide the Chemical Composition for impregnation via spraying into disposal surgical masks and surgical sleeved gowns to inactivate bacteria and enveloped viruses such as Coronavirus, SARS, MERS, Influenza A and Influenza B. The said chemical composition comprising of aqueous colloidal silver nanoparticles, Isopropanol Alcohol/Ethanol, Chlorhexidine gluconate/Quaternary Ammonium Compound (alkyl dimethyl benzyl ammonium chloride, alkyl didecyl dimethyl ammonium chloride, and dialkyl dimethyl ammonium chloride) and Peppermint/Spearmint Essential oils and water.

In an embodiment of the said invention, the aqueous colloidal silver nanoparticle is used as co-disinfectant and to enhance the excellent residual effect of antimicrobial properties of the disinfectant impregnated into the disposal surgical mask and sleeved gowns. The aqueous colloidal silver nanoparticle's antimicrobial effect has been demonstrated in numerous applications against different types of microorganisms. The mechanism behind the bactericidal efficacy of the aqueous silver nanoparticles is through its binding to disulphide or sulfhydryl groups in cell wall proteins. Silver nanoparticle also binds to DNA. Through these binding events, metabolic processes are disrupted, leading to cell death. The size of the colloidal silver nanoparticle ranges from 2-150 nm. The optimal size of spherical silver nanoparticles (AgNPs) for off-resonance surface-enhanced Raman scattering (SERS) was found to be ˜50 nm based on the equivalent Ag content in AgNP colloids. Hence, the preferred size of the colloidal size nano-size is 50 nm.

In another embodiment of said invention Isopropanol/Ethanol is used to increase the hydrophilic properties of the peppermint/spearmint essential oils and to facilitate the ease of said chemical composition to be impregnated into disposal surgical masks and sleeved gowns made from hydrophobic materials. It is also used for the denaturation of the capsid protein of the enveloped viruses such as Coronavirus, SRAS, MERS, Influenza A, Influenza B and for destroying the peptidoglycan/lipopolysaccharide cell wall of the bacteria. The preferred alcohol is Isopropanol Alcohol because it less dehydrating on living tissue and so is a better solution for disinfecting skin than ethanol. It is also effective against bacteria spores.

In yet another embodiment of the said invention, chlorhexidine gluconate/Quaternary Ammonium Compound (alkyl dimethyl benzyl ammonium chloride, alkyl didecyl dimethyl ammonium chloride, and dialkyl dimethyl ammonium chloride) is used as a co-disinfectant for the said chemical composition. Chlorhexidine gluconate is the preferred choice due to its a broad-spectrum biocide property and its effectiveness against Gram-positive bacteria, Gram-negative bacteria and fungi. It has excellent for both bacteriostatic (inhibits bacterial growth) and bactericidal (kills bacteria) making it an excellent choice for the said chemical composition used for the impregnation disposal surgical masks and sleeved surgical gowns.

In yet another embodiment of the said invention Peppermint/Spearmint essential oil is used to further enhanced the antimicrobial effect of the said invention. The preferred essential oil is peppermint, when used in a mixture of polar solvent such as Isopropanol Alcohol it is highly effective against Bacillus subtilis, Pseudomonas aeruginosa, Streptococcus aureus than Pseudomonas aureus and Serratia marcescens. Peppermint essential oil is also found to possess antiviral and fungicidal activities.

In yet another further embodiment, water is primarily used to as as a catalyst and has a major role in denaturing the proteins of cell membranes of viruses and bacteria. Also, the function of water slows down evaporation of said chemical composition and increases the surface contact time with the membrane and as a carrier for the all the above-mentioned ingredients of the said chemical composition. Preferred water is municipal fresh water due to its ease of access.

The detailed chemical composition for Impregnation via spraying on disposal surgical masks and sleeved Gowns made from Hydrophilic and Hydrophobic materials for inactivation bacteria and enveloped viruses such as Coronavirus, SARS, MERS, Influenza A and Influenza B is herein described:

1) In accordance with the invention, the main constituent of the chemical composition comprising 50-85% by weight of Isopropanol/Ethanol Alcohol, 0.1-8% by weight Chlorhexidine gluconate/Quaternary Ammonium Compound (alkyl dimethyl benzyl ammonium chloride, alkyl didecyl dimethyl ammonium chloride, and dialkyl dimethyl ammonium chloride) 5-30% by weight of aqueous colloidal silver nanoparticles 0.1-5% by weight of Peppermint/Spearmint essential oils and 2-17% by weight of water.

2) 50-85% by weight of Isopropanol Alcohol/Ethanol Alcohol is loaded to the mixing mixer tank to be mixed for 10-15 minutes with 2-17% by weight of water.

3) 0.1-8% by weight Chlorhexidine gluconate/Quaternary Ammonium Compound (alkyl dimethyl benzyl ammonium chloride, alkyl didecyl dimethyl ammonium chloride, and dialkyl dimethyl ammonium chloride) is then added in the above mixture in the mixing mixer tanks for 5-10 minutes.

4) 5-30% of colloidal silver nanoparticle is then added into said tank and mixed for 10-15 minutes.

5)) 0.1-5% by weight of peppermint/spearmint essential oil is then added into said tank and mixed for 15-20 minutes.

6) 2-17% by weight of water is then added into the said tank to be mixed for about 10 minutes and then chemical composition is ready to be used for impregnation of disposable surgical masks and sleeved gowns made from hydrophilic and hydrophobic materials.

### EXAMPLES

The following are the illustrative of various embodiments of the foregoing invention.

### Example 1

This example illustrates the chemical composition comprising of aqueous colloidal silver nanoparticles, Isopropanol Alcohol/Ethanol Alcohol, Chlorhexidine glucoante/Quaternary Ammonium Compound (alkyl dimethyl benzyl ammonium chloride, alkyl didecyl dimethyl ammonium chloride, and dialkyl dimethyl ammonium chloride), Peppermint/Spearmint Essential Oils and water used for impregnating disposal surgical masks and sleeved gowns made from hydrophilic and hydrophobic materials which typically consists of 20% by weight of aqueous colloidal silver nanoparticles, 70% by weight of Isopropanol Alcohol, 0.4 % by weight of Chlorhexidine gluconate 1% by weight of Peppermint oil and 8.6% by weight of water.

### Example 2

This example illustrates the chemical composition comprising of colloidal silver nanoparticle, Isopropanol/Ethanol Alcohol, Chlorhexidine/Quaternary Ammonium Compound, Peppermint/Spearmint Essential Oils and water used for impregnating disposal surgical masks and sleeved gowns made from hydrophilic and hydrophobic materials which typically consists 12% by weight of aqueous colloidal silver nanoparticles, 71% by weight of Isopropanol Alcohol, 1% by weight of Chlorhexidine gluconate, 1% by weight of Peppermint oil and 15% by weight of water.

### Example 3

This example illustrates the chemical composition comprising of colloidal silver nanoparticle, Isopropanol/Ethanol Alcohol, Chlorhexidine gluconate/Quaternary Ammonium Compound (alkyl dimethyl benzyl ammonium chloride, alkyl didecyl dimethyl ammonium chloride, and dialkyl dimethyl ammonium chloride), Peppermint/Spearmint Essential Oils and water used for impregnating disposal surgical masks and sleeved gowns made from hydrophilic and hydrophobic materials which typically consists of 10% by weight of aqueous colloidal silver nanoparticles, 70% by weight of Isopropanol Alcohol, 2% by weight of Chlorhexidine 1% by weight of Peppermint essential oil and 17% by weight of water.

1) The chemical composition suitable for impregnation of disposal surgical masks and sleeved gowns made from hydrophilic and hydrophobic materials for inactivation bacteria and enveloped viruses comprising of the synergistic chemical formulation.

2) The chemical composition suitable for impregnation of disposal surgical masks and sleeved gowns made from hydrophilic and hydrophobic materials for inactivation bacteria and enveloped viruses as in claimed 1 wherein the synergistic chemical formulation comprising of comprising of aqueous colloidal silver nanoparticles, Isopropanol Alcohol/Ethanol Alcohol, Chlorhexidine gluconate/Quaternary Ammonium Compound, Peppermint/Spearmint Essential oils and water.

3) The chemical composition suitable for impregnation of disposal surgical masks and sleeved gowns made from hydrophilic and hydrophobic materials for inactivation bacteria and enveloped viruses as in claimed 2 wherein aqueous colloidal silver nanoparticles is 5-30% by weight and with size ranges from 2-150 nm.

4) The chemical composition suitable for impregnation of disposal surgical masks and sleeved gowns made from hydrophilic and hydrophobic materials for inactivation bacteria and enveloped viruses as in claimed 2 wherein 50-85% by weight of synergistic chemical is Isopropanol/Ethanol Alcohol.

5) The chemical composition suitable for Impregnation of disposal surgical masks and sleeved gowns made from hydrophilic and hydrophobic materials for inactivation bacteria and enveloped viruses as in claimed 2 wherein 0.1%-8% by weight of the synergistic chemical is Chlorhexidine gluconate/quaternary ammonium compound.

6) The chemical composition suitable for impregnation of disposal surgical masks and sleeved gowns made from hydrophilic and hydrophobic materials for inactivation bacteria and enveloped viruses as in claimed 5, wherein, 0.1-8% by weight of the synergistic chemical formula is chlorhexidine gluconate.

7) The chemical composition suitable for impregnation of disposal surgical masks and sleeved gowns made from hydrophilic and hydrophobic materials for inactivation bacteria and enveloped viruses as in claimed 2, wherein, 0.1%-5 by weight of the chemical formula is Peppermint/Spearmint essential oil.

8) The chemical composition suitable for impregnation of disposal surgical masks and sleeved gowns made from hydrophilic and hydrophobic materials for inactivation bacteria and enveloped viruses as in claimed 2, wherein 2-17% by weight of the chemical formula is water.

9) The chemical composition suitable impregnation of disposal surgical masks and sleeved gowns made from hydrophilic and hydrophobic materials for inactivation bacteria and enveloped viruses as in claimed 2-8, wherein the said chemical formula increases the residual inactivation efficacy against enveloped viruses and bacteria of the disinfectant via its binding to disulphide or sulfhydryl groups in cell wall proteins where the metabolic processes are disrupted, leading to cell death.
